(19) United States
(12) Patent Application Publication
(10) Pub. No.: US 2025/0235329 A1
(43) Pub. Date: Jul. 24, 2025

(54) A MECHANICAL FINGER FOR AN ARTIFICIAL WRIST AND A MECHANICAL FINGER ACTUATOR

(71) Applicants: Dmytro GAZDA, Kyiv (UA); ESPER INC., Wilmington, DE (US)

(72) Inventors: Dmytro GAZDA, Kyiv (UA); Vitalii KUCHMA, Kramatorsk, Donetsk region (UA); Ihor ILCHENKO, Poltava (UA); Stanislav KOKIN, Polovynkyne, Luhansk region (UA)

(21) Appl. No.: 18/848,415

(22) PCT Filed: Jul. 7, 2022
(86) PCT No.: PCT/UA2022/000033

§ 371 Date: Sep. 18, 2024

(30) Foreign Application Priority Data
Mar. 18, 2022 (UA) A 2022 00979

(51) Int. Cl. A61F 2/58; A61F 2/50; A61F 2/70

(52) U.S. Cl. CPC A61F 2/586; A61F 2/70; A61F 2002/5043; A61F 2002/5073

(57) ABSTRACT

The claimed group of inventions relates to a field of anatomical engineering, and it relates to a mechanical finger and an actuator therefor, which may be used in imitators and prostheses of upper limbs. The mechanical finger is a structural element that performs a gripping function, consists of movable phalanxes, elements for modifying their position relative to each other, and a driving rope that is connected to a lead nut that is, in turn, intended to interact with an electromechanical actuator that transmits a force to the mechanical finger. According to the invention, the rope is fixed on the lead nut and divided into two portions which are arranged symmetrically relative to a sagittal plane of the proximal phalanx and laid on the guides. Therewith, cavities are provided in locations of the rope which are configured to receive loops of the rope, and a length of each of them equals to a travel length of the lead nut. The actuator is a power module that is aimed to drive and to control a movement of the mechanical finger. It consists of a motor reducer having a front shaft with a lead screw mounted thereon, the lead screw is configured to be coupled to the lead nut, and an encoder that is configured to enable a connection to the contact board of the artificial wrist, thereby powering the motor reducer and transmitting pulses. The actuator and the mechanical finger are individual modules of the artificial wrist which together form a single module.

### FIELD OF THE INVENTION

The claimed group of inventions relates to a field of anatomical engineering, preferably of a medical purpose, and it relates to a mechanical finger and an actuator therefor, which may be used as structural elements in imitators and prostheses of upper limbs.

### PRIOR ART

The prior art discloses devices which reproduce functions and appearance of a human hand. These devices may be used as limb imitators in robot technology for various purposes, e.g., in working manipulation processes, as well as in demonstration and educational projects. However, a preferable usage sector for them is medicine, and there is a high worldwide demand for creation of real, functional, as well as qualitative and available prostheses for disabled persons.

At present, one of the most completed types of upper limbs prosthetics is to create bionic prostheses which partially or fully replace an organ which is lost or absent at birth and perform its functions, and wherein one finger is usually operated by a separate drive (actuator) that is connected to a corresponding component of a prosthesis base and intended to convert signals received from myoelectric currents which are generated upon contraction of a human muscle or a group of muscles in order to receive command signals to bend or to unbend the finger.

This provides such prostheses with a significant advantage as compared to other types of the prostheses such as cosmetic or tension prostheses in terms of manipulations with items, since an operation system is based on squeezing or unsqueezing movements generated by pulses of a human body and enables to adjust forces resulting from these movements depending on their tasks.

The Applicant has chosen several technical solutions from prior art actuator-containing fingers for bionic artificial wrists, the solutions are close to the proposed invention in terms of a set of essential features.

A patent U.S. Pat. No. 6,896,704 B1 dated May 24, 2005 teaches a movable mechanical finger having an actuator for upper limb prostheses. According to this technical solution, the mechanical finger comprises a base, a first intermediate portion connected to the base, a second intermediate portion connected to the base, and a fingertip that is connected to the second intermediate portion. Bending of separate portions of the mechanical finger is performed by means of a system of wires which are secured around pulleys and passed through an inner portion of the finger, while unbending is performed by means of tension springs which are secured between the finger portions above them. The actuator is mounted in the finger base between the base and the first portion, and it is connected to a wire that is arranged on a drive shaft of the actuator by an eccentric member. The technical solution implies use of a linear actuator that comprises an electric servomotor having an encoder, a screw shaft that is mounted on the base such that it is capable of performing a circular rotation and that is coupled to an output shaft of the servomotor via a combination of gears which act as a worm gear, and a slider that is engaged to the screw shaft and slidably mounted within the base and connected to the wires. A drawback of this solution lies in a provision of the gear on the output shaft of the servomotor, the gear is characterized by an insufficient movement smoothness and a risk of jamming of the gears, as well as in a complexity of the tension system in a form of bypass wires which pass through the entire finger and several springs, which may lead to an inoperability of the bending mechanism of individual portions of the finger in case of failure, deformation or fracture of at least one of the wires and/or springs. One more drawback lies in that the actuator is designed as an integral portion of the finger, thereby requiring the whole assembly to be replaced in case of failure of the actuator or the finger elements.

Also, a finger prosthesis according to an application WO2021095014 A1 dated May 20, 2021 is known, the finger prosthesis comprises an actuator which contains a housing, a motor that is arranged within the housing, an output shaft having a worm gear that passes proximally along a rotation axis, wherein the motor is mechanically coupled to the output shaft and is configured to cause a rotation of the output shaft around a rotation axis, a first bearing that is arranged on a proximal end of the output shaft proximally relative to the worm gear, a second bearing that is arranged on a distal end of the output shaft distally relative to the worm gear, and a worm wheel that is configured to be fixed to a hand prosthesis. Therewith, the worm wheel is mechanically coupled to the worm gear such that the worm gear rotates along the worm wheel, thereby causing, in turn, a rotation of the motor and of the housing around the worm wheel. According to the proposed technical solution, the finger prosthesis further comprises a rope- or wire-like element that may be made elastic or non-elastic and that passes from the worm wheel or nearby along the housing of the mechanical finger to its distal segment and a tension spring which force proximal and distal segments of the finger to open and to close, when the worm gear moves along the worm wheel, wherein the tension spring shifts the finger, thereby opening it, and the wire-or rope-like element generates a traction force in order to close the finger. A drawback of this technical solution lies in that the actuator is made using the worm gear that is prone to be worn quickly and, thus, in that a frequent repairing is needed. Since the worm gear mechanism is characterized by increased requirements as to assembly and adjustment accuracy, this complicates a process of manufacturing the prosthesis, while the fixation of the actuator to the hand prosthesis by means of the worm wheel complicates a possible replacement of the actuator. Furthermore, a use thereof is characterized by an insufficient movement smoothness and by a high probability of jamming of the gears, thereby negatively affecting a uniformity of transmission of a force to the finger from the actuator and complicating the use of the prosthesis in general.

A patent U.S. Pat. No. 11,007,071 B2 dated May 18, 2021 discloses a prosthesis comprising a finger portion and an actuator unit for the finger portion, wherein the actuator comprises an output portion of the actuator that is a rotor or a linear motor; a movable portion that is configured to be connected to the output portion of the actuator and that is movable under action of the output portion of the actuator; a drive rope that is configured to be arranged within the finger portion and that is connected to a lead screw of the movable portion of the actuator and that drives the finger portion of the prosthesis. The actuator unit further comprises a rope-shaped elastic member that is configured to return the finger into an initial state under an action of elasticity and that is arranged within the finger portion in parallel to the drive rope, wherein the finger consists of at least two portions and comprises at least one pivot portion, and adjacent portions of the finger are pivotally connected between each other via the pivot portion, the drive rope bypasses the pivot portion from below and it is arranged around protruding guiding shafts, while the elastic rope passes above the pivot portion. Contacts of the actuator are connected to a processor within a housing portion of the hand prosthesis, the processor processes signals from myoelectric sensors, controls the rotation of the rotor of the motor that, in turn, controls the finger movement. A drawback of the proposed technical solution lies in that the drive rope is arranged along the entire finger portion of the prosthesis around several guiding shafts which may lead to a damage of the rope as a result of friction forces which occur during its sliding around the guiding shafts, while use of the elastic rope to return the finger portion to a rectified state is not effective enough as compared to, e.g., use of tension springs which are characterized by increased reliability and durability. Furthermore, the prosthesis structure does not enable to perform an assembly repairing of individual modules comprised therein, which is not rational and is not convenient for a user.

A mechanical finger for prosthetics and gripping devices according to a patent U.S. Pat. No. 10,426,636B2 dated Oct. 1, 2019 is taken as the closest analogue, the finger comprises a base; a proximal phalanx that is hingedly connected to the base and a distal phalanx that is hingedly connected to the proximal phalanx, which are connected between each other by means of elements for modifying their mutual position and which are driven by a flexible traction that is coupled to the drive. An electromechanical actuator coupled to the flexible traction is used as the drive for modifying the position of the proximal phalanx relative to the base and of the distal phalanx relative to the proximal one. According to the technical solution, the actuator is arranged directly in the proximal phalanx, a worm gear is used as the element for coupling the actuator to the flexible traction, while the flexible traction itself that may be designed as a belt, a fiber part, a strand, a synthetic thread, a string or a flexible wire, fixed on the base, and passes through both phalanxes, in particular, under an axis of rotation of the distal phalanx, along a system of guiding elements, to enable loading by a force acting from elements of mutual position of the phalanxes.

A drawback of the proposed technical solution lies in a complexity of the mechanical finger structure that is characterized by a presence of a large number of intermediate parts to transfer forces from the actuator to the phalanxes that, in turn, increases a probability of failure of the prosthesis upon damage or deformation of any of the elements of its structure, e.g., one of the spring elements to return the proximal and/or the distal phalanx into an initial position or the tension spring associated with a guiding roller. The structure of the finger does not. enable to conduct an assembly repairing, since the actuator is arranged directly in the proximal phalanx. Furthermore, mounting of the finger together with the actuator into the prosthesis implies its implantation into an osteointegrated component of the base, i.e., a non-releasable connection to the actuator's contacts, which, in case of damage of the mechanical finger, will lead to a situation in which the user would remain without the prosthesis for a long period of time in order it could be fully repaired.

Therefore, main drawbacks of the presently known bionic prostheses lie either in their monolithic nature, thereby complicating their repairing or replacement, or in a presence of many branched elements for bending-unbending and elements for driving thereof, thereby implying a complex adjustment, fitting, building movement trajectories etc., and negatively affects a uniformity of the force transfer to the finger from the actuator, and, thus, impairs the smoothness and accuracy of the movements. Furthermore, the known solutions do not provide a protection of the operative mechanisms of the finger both as a separate module and being coupled to the actuator against an influence by external loads that may arise, e.g., as a result of falling or beating and negatively affect the correct operation of the prosthesis in general.

### SUMMARY OF THE INVENTION

The group of inventions is based on a task to create a reliable mechanical finger having a simple structure and an optimal number of elements for enabling its bending-unbending and a protection of their operation against influencing by external loads, and an actuator to drive the finger in a defined position and to provide a uniform transfer of a force from the actuator to the mechanical finger, as well as to provide a modularity of the structure of the mechanical finger and of the actuator which forms their functional integrity upon their combination.

According to a first embodiment of the claimed invention, the posed task is resolved by the fact that the mechanical finger for an artificial wrist consists of at least two movable phalanxes, a proximal one and a distal one, which are hingedly connected between each other and coupled by elements for modifying a position of the distal phalanx relative to the proximal phalanx, which are driven by means of a flexible traction. The proximal phalanx is hingedly connected to an immovable base that is configured to be coupled to an electromechanical actuator and is equipped with an element for providing an interaction between the actuator and the flexible traction that is made in a form of a rope. According to the claimed embodiment of the invention, a lead nut is used as the element for providing the interaction between the actuator and the flexible traction, and the lead nut is arranged outside the hinge connection between the base and the proximal phalanx and is equipped with a fixation element to avoid its rotation. A rope is used as the flexible traction, the rope is secured on the lead nut and divided into two portions which are arranged symmetrically relative to a sagittal symmetry plane of the proximal phalanx and are laid on guides, wherein first of them are arranged on the base and represent bypass supports, while other ones represent curved guides which are provided in the proximal phalanx around an axis of the hinge connection. Therewith, cavities are provided between the base and the proximal phalanx, the cavities are coupled to the curved guides so as they can receive loops of the rope, and a length of each of them equals to a travel length of the lead nut. Ends of the rope are fixed in the proximal phalanx. By making the lead nut as a constituent part of the mechanical finger arranged outside the hinge connection of the base and connected to the proximal phalanx by means of two symmetrically arranged portions of the rope, a modularity of the structure of the claimed mechanical finger is provided, thereby enabling to perform an easy and a quick replacement thereof independently of the actuator. By making the flexible traction of the rope having portions which are arranged symmetrically relative to the sagittal plane of the proximal phalanx, a uniform transfer of forces from the actuator is provided and bending of the proximal phalanx with the same force is enabled, thereby increasing an operation reliability of the claimed mechanical finger and increasing a functionality thereof. Furthermore, provision of the curved guides for arranging the rope in the proximal phalanx enables to arrange the rope directly in a first joint of the mechanical finger that is formed by the base and the proximal phalanx and cavities coupled thereto which are arranged between the base and the proximal phalanx so as they can receive the loops of the rope, each of them has the length that equals to the travel length of the lead nut, enables to provide the smoothness of running of the proximal phalanx and the distal phalanx associated therewith, as well as to implement a passive bending of the finger phalanxes when applying external forces thereto without the actuator's influence, thereby, in turn, minimizing a risk of damage and, thus, of an incorrect operation of the finger upon falling or beating of the same.

According to one of exemplary embodiments, a rigid traction in a form of a lever having ends which are secured on rotation axes in the distal phalanx and in the base, and an elastic reverse force element in a form of a tension spring having ends which are secured in the distal phalanx and in the proximal phalanx respectively, are used as the elements for modifying the position of the distal phalanx relative to the proximal phalanx.

By making the rigid traction in the form of the lever, bending of the distal phalanx together with the proximal phalanx during movement of the lead nut in a proximal direction is provided, while the presence of the elastic element in the form of the tension spring in the structure of the mechanical finger, the tension spring is characterized by a structural simplicity and by a capability of being elastically and significantly deformed, and the deformations are maintained stable during a long time period, the return of the finger into the initial (unbent) position during the movement of the lead nut in a distal direction is provided. In should be appreciated that any other element having elastic properties may be used instead of the tension spring, e.g., an elastic rope or a torsion spring, a cable etc., without falling beyond the concepts of the present invention.

According to a further exemplary embodiment of the invention, a bearing that is mounted on an external surface of the nut is used as an element for fixation of the lead nut to avoid its rotation, wherein an axis of the bearing is perpendicular to the axis of the nut. Use of the element for fixation of the lead nut to avoid its rotation avoids its non-controlled circular rotation around its own axis, thereby significantly increasing a general operation efficiency of the mechanical finger due to avoiding a possible disordered movement of the finger phalanxes, while by making the element for fixation of the lead nut to avoid its rotation in the form of the bearing that is mounted on the external surface of the nut such that the bearing axis is perpendicular to the axis of the nut, the reliability of such fixation is provided.

Therewith, the curved guides are made of metal, thereby avoiding a damage of an internal surface of the housing of the proximal phalanx under action of a pressure onto the guides that is caused during movement of the rope in the proximal or distal directions.

Therewith, according to one of exemplary embodiments, the bypass supports are made as roller supports, thereby providing a smooth and noiseless movement of the ropes.

Therewith, the distal and/or the proximal phalanxes have external envelopes to strengthen the structure of the mechanical finger, to improve the engagement with items during gripping, and to improve its visual perception due to a possible various designing.

According to a second embodiment of the claimed invention, the posed task is resolved by the fact that the mechanical finger for an artificial wrist consists of at least two movable phalanxes, a proximal one and a distal one, which are hingedly connected between each other and coupled by elements for modifying a position of the distal phalanx relative to the proximal phalanx, which are driven by means of a flexible traction, wherein the proximal phalanx is hingedly connected to an immovable base that is configured to be coupled to an electromechanical actuator and is equipped with an element for providing an interaction between the actuator and the flexible traction. According to the invention, a lead nut is used as the element for providing the interaction with the flexible traction, and the lead nut is arranged outside the hinge connection between the base and the proximal phalanx and is equipped with a fixation element to avoid its rotation. A rope is used as the flexible traction, the rope is fixed on the nut and divided into two portions which are arranged symmetrically relative to a sagittal plane of the proximal phalanx and laid on the guides. The first guides are immovably secured on the base and represent bypass supports. The second guides for the rope are arranged within the hinge connection that is formed by the base with a rotor mounted thereon, the rotor is immovably connected to the proximal phalanx, and they represent bypass supports secured on the rotor. Therewith, a travel of the bypass supports of the rotor is limited by a chamber formed in the base, the chamber is configured to receive loops of the rope, and a length of each of them equals to a travel length of the lead nut. Ends of the rope are fixed in the base.

By making the lead nut as a constituent part of the mechanical finger arranged outside the hinge connection of the base and connected to the base by the rope, a modularity of the structure of the claimed mechanical finger is provided, thereby enabling to perform an easy and a quick replacement thereof independently of the actuator. By arranging the portions of the rope symmetrically relative to the sagittal plane of the proximal phalanx, a uniform transfer of forces from the actuator is provided and bending of the proximal phalanx with the same force is enabled, thereby increasing an operation reliability of the claimed mechanical finger and increasing a functionality thereof. The possibility of arrangement of the rope loops in the chamber formed in the base, where each of them has the length that equals to the travel length of the lead nut, enables to implement a passive bending of the phalanxes when applying external forces thereto without involving the actuator, thereby, in turn, minimizing a risk of damaging and, thus, of incorrect operation of the prosthesis upon falling or beating of the same. By making the guides in the form of the bypass supports which are arranged on the base and bypass supports arranged on the rotor, while their travel is limited by the chamber that is formed in the base, it is enabled to provide smoothness, noiselessness, and uniformity of movement of the rope portions, and, thus, of the proximal phalanx and of the distal phalanx associated therewith, thereby enabling to achieve a maximum anatomy of the claimed mechanical finger and its similarity to a biological finger of a human wrist in terms of its functionality.

According to one of exemplary embodiments, a rigid traction in a form of a lever having ends which are secured on rotation axes in the distal phalanx and in the base, and an elastic reverse force element in a form of a tension spring having ends which are secured in the distal phalanx and in the proximal phalanx respectively, are used as the elements for modifying the position of the distal phalanx relative to the proximal phalanx. By making the rigid traction in the form of the lever, bending of the distal phalanx together with the proximal phalanx during movement of the lead nut in a proximal direction is provided, while the presence of the elastic element in the form of the tension spring in the structure of the mechanical finger, the tension spring is characterized by a structural simplicity and by a capability of being elastically and significantly deformed, and the deformations are maintained stable during a long time period, the return of the finger into the initial (unbent) position during the movement of the lead nut in a distal direction is provided. In should be appreciated that any other element having elastic properties may be used instead of the tension spring, e.g., an elastic rope, a cable etc., without falling beyond the concepts of the present invention.

According to an exemplary embodiment of the invention, locations for arranging the ropes are closed by covers secured to the base which act as rotor sleeves, wherein it rotates, assist in protecting the rope locations against penetration of dust or moisture and avoid a contact of the rope with the proximal phalanx.

According to an exemplary embodiment of the invention, a bearing that is mounted on an external surface of the nut is used as an element for fixation of the lead nut to avoid its rotation, wherein an axis of the bearing is perpendicular to the axis of the nut. Use of the element for fixation of the lead nut to avoid its rotation avoids its non-controlled circular rotation around its own axis, thereby significantly increasing a general operation efficiency of the mechanical finger due to avoiding a possible disordered movement of the finger phalanxes, while by making the element for fixation of the lead nut to avoid its rotation in the form of the bearing that is mounted on the external surface of the nut such that the bearing axis is perpendicular to the axis of the nut, the reliability of such fixation is provided. Furthermore, protrusions or recesses provided on the external surface of the nut or any other equivalent fixation elements, without falling beyond the concepts of the present invention, may be used as the fixation elements.

Therewith, according to one of exemplary embodiments, the bypass supports are made as roller supports, thereby providing a smooth and noiseless movement of the ropes.

Therewith, the distal and/or the proximal phalanxes have external envelopes to strengthen the structure of the mechanical finger and to improve its visual perception due to a possible various designing.

By making the invention according to both the first and the second embodiments enables to create the mechanical finger being the closest one, in terms of anatomy, to the biological human finger which completely reproduces the functionality of the latter and represents the reliable and accurate execution mechanism for gripping items which have even the smallest dimensions.

A further subject matter of the group of inventions is an electromechanical actuator to drive and to control the mechanical finger. The actuator comprises a motor reducer having a front shaft with a lead screw mounted thereon that is configured to be coupled to the lead nut of the mechanical finger, wherein a portion of the motor reducer that is coupled to the lead screw is arranged in the housing, while its output portion forms a chamber of the lead screw, the chamber is equipped with a guide for linear movement of the element for fixation of the lead nut to avoid its rotation and is configured to be connected to the finger's base. Therewith, an encoder is connected to an opposite portion of the motor reducer, the encoder comprises a plate that is mounted on the motor reducer, and a motor reducer board that is connected to contacts of the motor reducer is mounted on the plate, the board is equipped with at least two Hall sensors which are displaced radially from each other by 90 angle degrees, and a magnet that is mounted on a rear shaft. Therewith, the encoder board is equipped with a means for a releasable electrical connection to corresponding contacts of the artificial wrist.

Use of the motor reducer in the actuator provides a series of advantages mainly including a high performance, a service simplicity, and a compactness. Use, in the chamber, of the lead screw of the guide for the linear movement of the element for fixation of the lead nut to avoid its rotation, thus, provides the uniformity of the transfer of forces to the finger from the actuator. By means of the encoder that reads a number of revolutions of the motor by means of the Hall sensors, the linear movement of the lead nut on the lead screw is calculated which is used to determine a bending angle of the mechanical finger, thereby providing the effective control of the latter. An advantage of use of the magnetic Hall sensors lies in their wear resistance due to absence of movable parts, rigidity, and almost complete lack of a maintenance need, as well as vibration, dust and water insensitivity. It should be noted that any other similar sensors may be used instead of the Hall sensors without falling beyond the concepts of the present invention. The provision of the encoder board with the means for the releasable electrical connection to the corresponding contacts of the artificial wrist provides the actuator's modularity and allows to carry out its easy replacement by performing easy operations.

Therewith, according to one of exemplary embodiments, the guide for the linear movement of the element for fixation of the lead nut to avoid its rotation is a longitudinal groove. It should be appreciated that a shape of the guide depends on a design shape of the element for fixation of the lead nut to avoid its rotation, and it also may be made in a form of a protrusion or slots etc.

Therewith, the means for the releasable electrical connection of the encoder board may be made in a form of saddles for needle contacts or flat platforms for spring-loaded contacts, thereby providing its connection to a contact board that is arranged, e.g., within a frame of the artificial wrist, depending on a modification thereof.

### DESCRIPTION OF THE DRAWINGS

In order to provide more complete understanding of the claimed invention and advantages thereof, the following description provides an explanation of possible exemplary embodiments thereof with a reference to figures of the appended drawings, wherein identical designations denote identical parts, and which illustrate the following:

FIG. 1 illustrates an exploded illustration of the structure of the mechanical finger according to the 1st embodiment of the invention;

FIG. 2 illustrates an axonometric partial cross-section of the mechanical finger according to the 1st embodiment of the invention;

FIG. 3 illustrates a longitudinal cross-section of the hinge connection between the base and the proximal phalanx of the mechanical finger according to the 1st embodiment of the invention;

FIG. 4 illustrates a longitudinal cross-section of the connection between the actuator and the mechanical finger according to the 1st embodiment of the invention in the operative state;

FIG. 5 illustrates a longitudinal cross-section of the connection between the actuator and the mechanical finger according to the 1st embodiment of the invention during the passive bending;

FIG. 6 illustrates an exploded illustration of the structure of the mechanical finger according to the 2nd embodiment of the invention;

FIG. 7 illustrates an axonometric partial cross-section of the mechanical finger according to the 2nd embodiment of the invention;

FIG. 8 illustrates an axonometric partial cross-section of the hinge connection between the base and the proximal phalanx of the mechanical finger according to the 2nd embodiment of the invention;

FIG. 9 illustrates a longitudinal cross-section of the hinge connection between the base and the proximal phalanx in the operative position of the mechanical finger according to the 2nd embodiment of the invention;

FIG. 10 illustrates a longitudinal cross-section of the hinge connection between the base and the proximal phalanx during the passive bending of the mechanical finger according to the 2nd embodiment of the invention;

FIG. 11 illustrates a longitudinal cross-section of the connection between the actuator and the mechanical finger according to the 2nd embodiment of the invention in a static state;

FIG. 12 illustrates a longitudinal cross-section of the connection between the actuator and the mechanical finger according to the 2nd embodiment of the invention in a dynamic state;

FIG. 13 illustrates a longitudinal cross-section of the connection between the actuator and the mechanical finger according to the 2nd embodiment of the invention during the passive bending;

FIG. 14 illustrates a general view of the actuator with a partial cross-section of the housing and with the contacts of the base of the artificial wrist;

FIG. 15 illustrates a detailed view of the encoder of the actuator;

FIG. 16 illustrates a view of the actuator with a partial cross-section of the housing;

FIG. 17 illustrates a general view of the actuator when combined with the finger;

FIG. 18 illustrates the modularity and the possibility of connection between the mechanical finger, the actuator, and the base component of the artificial wrist.

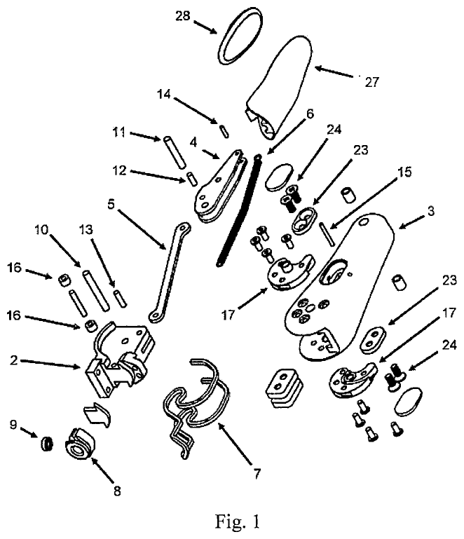

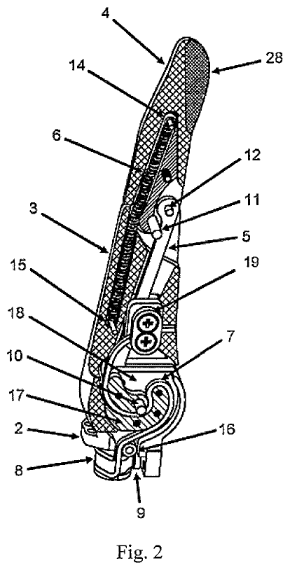

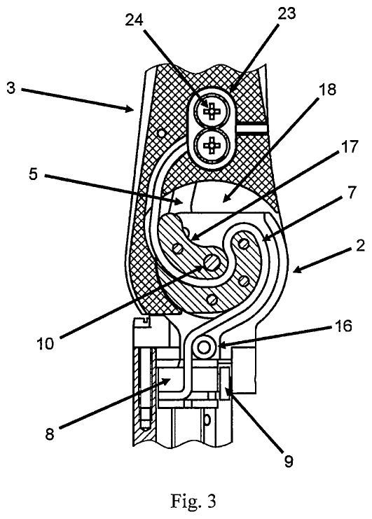

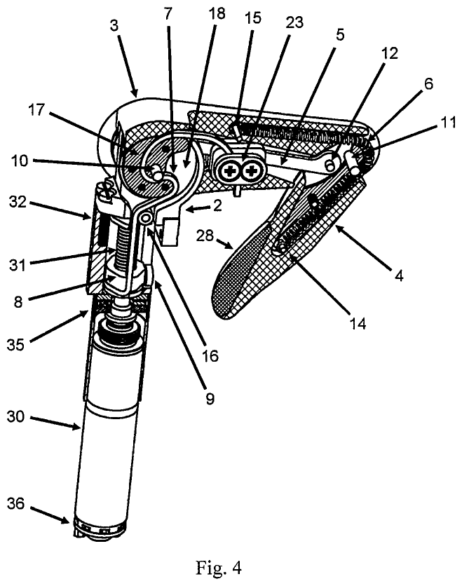

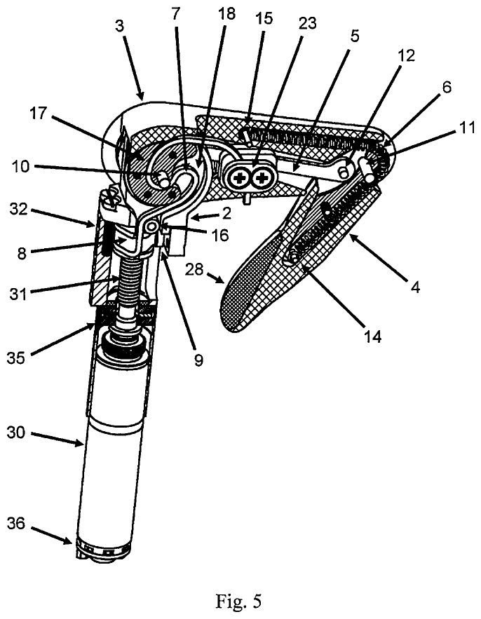

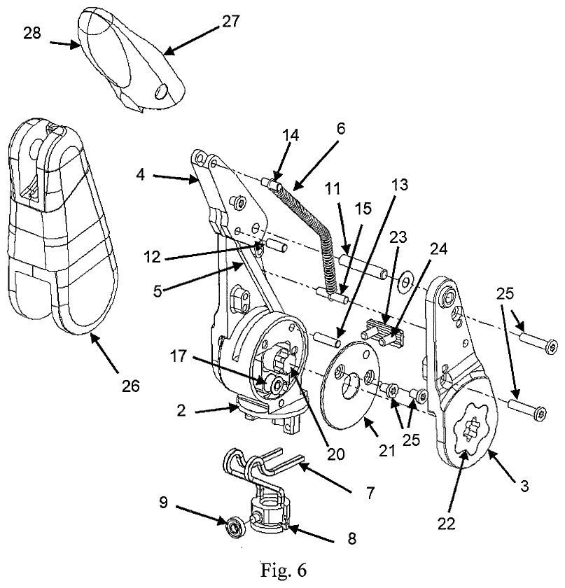

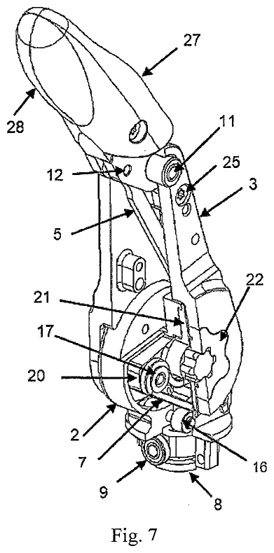

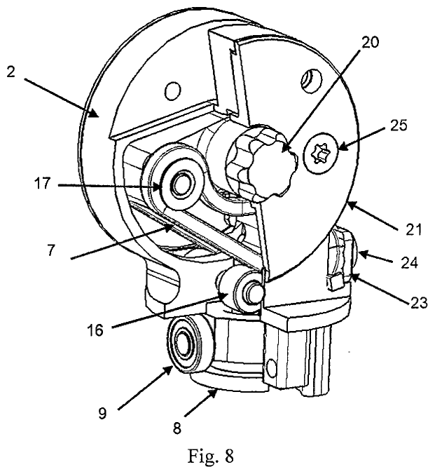

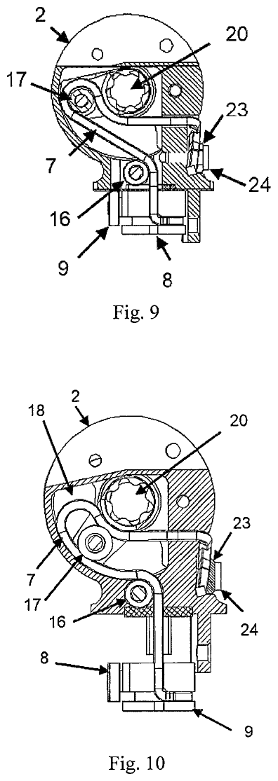

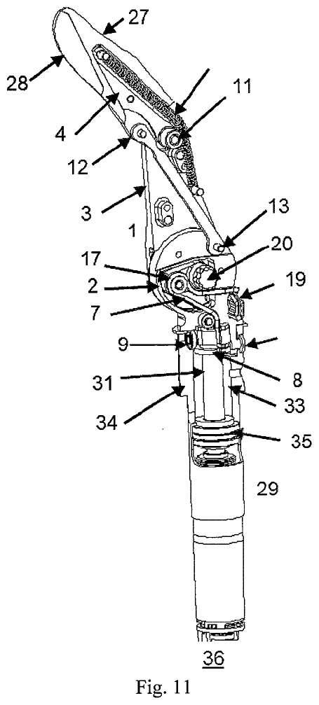

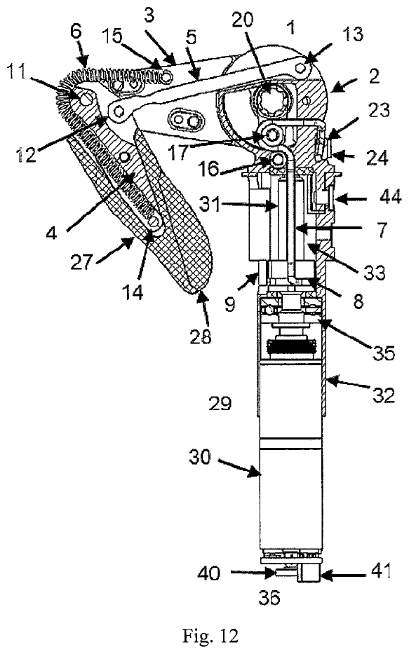

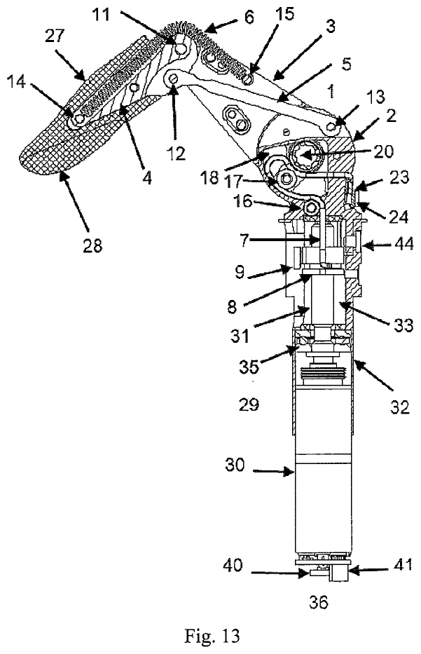

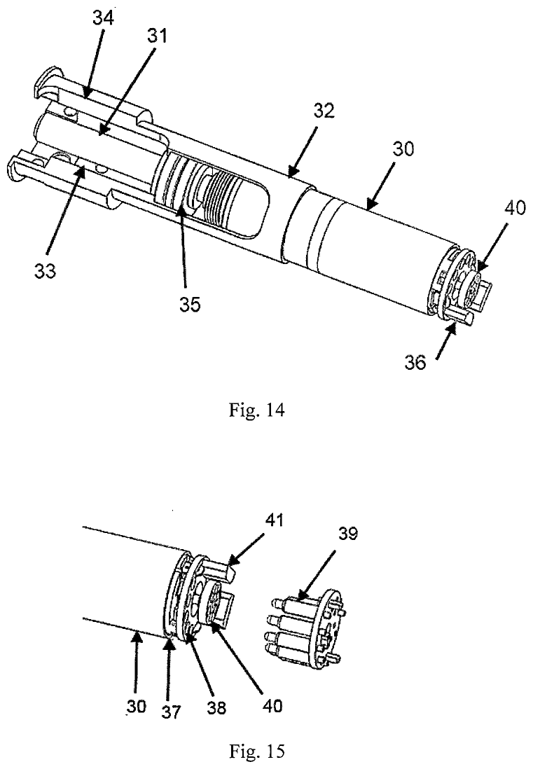

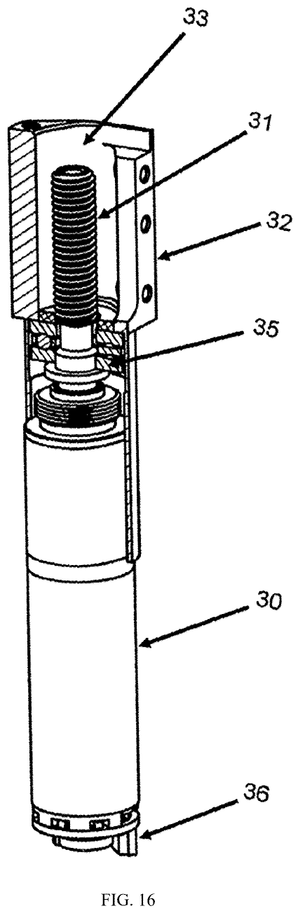

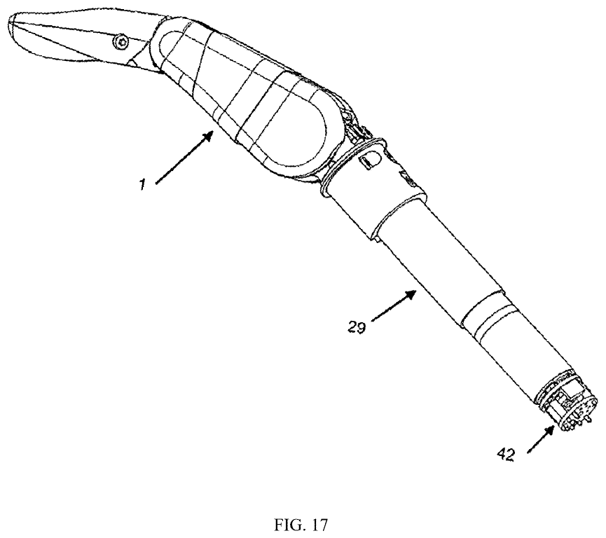

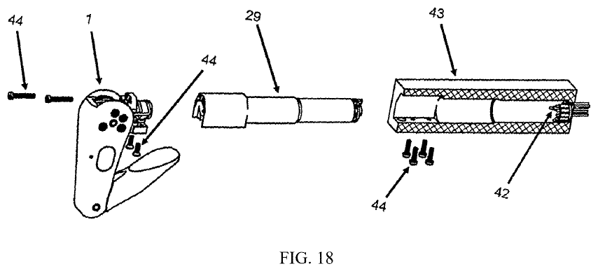

### MAIN DESIGNATIONS

1. Finger
2. Base
3. Proximal phalanx
4. Distal phalanx
5. Rigid traction
6. Reverse force spring
7. Rope
8. Lead nut
9. Element for fixation of the lead nut to avoid its rotation
10. Axis of the hinge connection between the base and the proximal phalanx
11. Axis of the hinge connection between the proximal phalanx and the distal phalanx
12. Pivot fixation axis of the rigid traction in the distal phalanx
13. Pivot fixation axis of the rigid traction in the base
14. Fixation site of the spring in the distal phalanx
15. Fixation site of the spring in the proximal phalanx
16. Bypass supports of the rope on the base
17. Guides of the rope within the hinge connection between the base and the proximal phalanx
18. Cavity for the loop of the rope
19. Fixation site of the rope ends
20. Rotor
21. Cover of the rotor
22. Insert
23. Clamp of the rope ends
24. Screw of the clamp of the rope ends
25. Fixation screws
26. External envelope of the proximal phalanx
27. External envelope of the distal phalanx
28. Pad of the distal phalanx
29. Actuator
30. Motor reducer
31. Lead screw
32. Housing of the actuator
33. Chamber of the lead screw
34. Guide
35. Thrust bearing of the lead screw
36. Encoder
37. Plate of the encoder
38. Board of the encoder
39. Electrical contact means
40. Magnet
41. Hall sensors
42. Contact board of the artificial wrist
43. Frame of the artificial wrist
44. Fixation screws

### IMPLEMENTATION POSSIBILITY

A mechanical finger 1, according to the claimed invention, consists of main elements being an immovable base 2, movable phalanxes being a proximal one 3 and a distal one 4, and elements for moving the phalanxes being elements 5, 6 for modifying a position of the distal phalanx 4 relative to the proximal phalanx 3, and a rope 7 for modifying a position of the proximal phalanx 3 relative to the base 2 and, thus, to drive the elements 5, 6 for modifying the position of the distal phalanx 4, as well as a lead nut 8 that is coupled to the rope 7 and equipped with a fixation element 9 to avoid its rotation, thereby providing its linear movement outside the finger 1. The base 2 together with the proximal phalanx 3 are mounted on a common axis 10, thereby forming a hinge connection, while the distal phalanx 4 together with the proximal phalanx 3, in turn, form a hinge connection around a common axis 11.

The element 5 is a rigid traction in a form of a lever having one end that is secured on a rotation axis 12 in the distal phalanx 4, while another end is secured on a rotation axis 13 that is provided in the base 2 within its hinge connection to the proximal phalanx 3, and it is intended to bend the phalanx 4 relative to the phalanx 3. The element 6 is an elastic reverse force element acting on the phalanx 4 to unbend it and it may be made in a form of a tension spring having ends which are secured on corresponding axes 14, 15 in the distal phalanx 4 and in the proximal phalanx 3.

The rope 7 is divided into two portions which are symmetrically arranged relative to a sagittal plane of the proximal phalanx 3 and laid on guides. First guides of the rope portions are immovably mounted on the base by means of bypass supports 16 which, according to the illustrated exemplary embodiments, are roller supports, while other guides 17 are provided within the hinge connection between the base 2 and the proximal phalanx 3 and are intended to provide a movement of the rope portions along a curved trajectory. Cavities 18 are provided within said hinge connection which are intended to receive protruding loops of the rope 7, if they are formed, and have a length that equals to a travel length of the lead nut 8. The length of the rope is selected considering a full travel made by the nut in order to achieve a maximum bending of the proximal phalanx 3 relative to the base 2. Therewith, the ropes are arranged in a straight line between the rope fixation site on the nut 8 and the bypass supports 16, and then they are brought within the hinge connection towards the bending of the finger, while bypassing around the axis of the hinge connection, and their ends are directed towards the unbending of the finger.

The guides 17 provide the rope 7, upon application of a traction force from the lead nut 8 thereto, with a synchronous movement of its parts along the curved trajectory and drive the proximal phalanx 3 relative to the base 2 by involving firstly the rigid traction 5 in order to drive the distal phalanx 4, thereby providing the bending movement of the finger 1.

During the reverse movement of the nut 8, the rope 7, while returning to the initial arrangement state together with the action of the reverse force spring 6, loosens the influence made by the rope 7 traction onto the proximal phalanx 3, thereby providing the unbending movement of the finger 1.

When applying external bending forces, e.g., a beat, onto the finger 1, an excess of the rope 7 will be formed within the hinge connection, and the excess protrudes outside the guides 17 into the cavities 18 in a form of two loops, thereby providing the bending of the proximal phalanx 3 relative to the base 2 and without moving the lead nut 8. A size of the cavity 18 matches with the length of the loops of the rope 7 which could be formed when the nut 8 is in a first extreme position without applying a traction force to the rope 7, i.e., they equal to the travel length thereof.

Such a design enables to drive all the finger elements with a uniform force, when the rope 7 travels only in a single joint of the mechanical finger that is formed by the hinge connection between the base 2 and the proximal phalanx 3, and to perform a passive bending of the finger phalanxes when applying external forces thereto without shifting the lead nut 8 relative to its current position and without a risk of a failure of the finger 1 elements.

Peculiarities of the first embodiment of the mechanical finger lie in that the guides 17 are provided in the proximal phalanx 3 in a form of curved channels around the axis 10, while the ends of the rope are brought outside the hinge connection and fixed in the proximal phalanx 3. Therewith, the cavities 18 for laying the loops of the rope are formed between the base 2 and the proximal phalanx 3 within the hinge connection therebetween in the distal direction.

Owing to this design, when applying the traction force to the rope 7 from the side of the lead nut 8, the rope 7 will travel along the curved trajectory forming a reverse bypass of the hinge connection axis, thereby driving directly the proximal phalanx 3 in the bending direction that, in turn, will drive the distal phalanx 4 affecting the pivot of the lever of the rigid traction 5 around the axis 13 within the immovable base 2.

Peculiarities of the second embodiment of the mechanical finger lie in that the transfer of the force from the rope 7 to the proximal phalanx 3 occurs indirectly via a rotor 20 mounted in the base. The guides 17 are provided in the base 2 and made as bypass supports on the rotor 20 that is immovably connected to the proximal phalanx 3. The function of the cavity 18 for the loops of the rope is performed by the chamber provided in the base 2, the chamber defines and limits a pivot of the rotor 20 and of the bypass supports 17 within maximum 100 angle degrees which corresponds to the required bending of the proximal phalanx 3. Therewith, the fixation site of the rope ends 19 is located within the base 2.

In a preferable exemplary embodiment of the invention, covers 21 are arranged between the base 2 and the proximal phalanx 3, the covers are secured to the base 2 and act as sleeves for the rotor in which it rotates, assist in protecting the locations of the rope against dust or moisture penetration, and which also avoid a contact between the rope and the proximal phalanx, thereby increasing the whole wear resistance of the structure.

With such design, during the tension of the rope 17, the rotor 20 pivots and the rope, while moving through a system of the guides 16, 17 and along an inner portion of the covers 21, transmits a rotation torque to the proximal phalanx 3 via the rotor 20 according to a tackle principle.

When the external bending force acts on the finger, the proximal phalanx 3 will return together with the rotor 20 and with the guides 17 relative to the base 2 and the rope loop formed in this case will be located in the cavity 18 without influencing the position of the lead nut 8.

Depending on dimensions of the finger and in order to provide the required bending angle, to transmit the torque from the rotor 20 to the proximal phalanx 3, the rotor 20 may be coupled to the phalanx via an insert 22.

In the illustrated exemplary embodiments of the invention, the fixation of the ends of the rope 7 in the proximal phalanx 3, according to the first embodiment of the invention, or in the base 2, according to the second embodiment, is provided by screws having clamps 23, 24, but it may be provided by another connection that is suitable for this purpose.

In the illustrated exemplary embodiments of the invention, the fixation element 9 of the lead nut 8 is depicted as a radial sliding bearing, however, it would be clear for a skilled person that other means suitable for this purpose may be used instead of it being protrusions or recesses provided on the external surface of the nut or fixation elements which are equivalent thereto, without falling beyond the concepts of the present invention.

The base 2 and the phalanxes 3, 4 may be made of any material that is suitable for this purpose being metal, plastics, composite, by means of casting, 3D-printing etc. A core of the proximal phalanx may be either a solid one having a site provided to receive the base therein or it may consist of two side pieces connected by means of fixation means 25. Therewith, according to one of examples of the first embodiment of the invention, the curved guides 17 of the proximal phalanx 3 are made of metal that will provide the increased wear resistance of the portion that is in contact with the rope 7, if the phalanx is made of a less rigid material.

In turn, the distal phalanx 4 also may have a solid or a collapsible frame, e.g., in a form of a bracket.

Both phalanxes or the distal phalanx only may have external envelopes 26, 27, while the envelope 27 of the distal phalanx 4 may have a finger pad 28. The envelopes are intended to improve the engagement with items during gripping, and they represent protective and decorative elements which strengthen and provide a real visual appearance of the finger, and they may be made of a suitable resin, silicone, polyurethane material etc.

In order to drive and to control the mechanical finger 1, an electromechanical actuator 29 is provided, which comprises a power unit in a form of a motor reducer 30 that is coaxial and preferably cylindrical, a mechanical portion in a form of a lead screw 31 mounted on a front shaft of the motor reducer, and an electrical portion in a form of the encoder 36 coupled to contacts of the motor reducer 30 from the opposite side.

The lead screw 31 is configured to be coupled to the lead nut 8, thereby providing a conversion of its rotational movement into a linear movement of the lead nut 8 from which the traction force is transmitted to the rope 7 in order to drive the mechanical finger 1. At least a portion of the motor reducer 30 is arranged in the housing 32, and an output portion thereof forms the chamber 33 of the lead screw 31. A guide 34 of the element 9 for fixation of the lead nut 8 to avoid its rotation is provided in the chamber 33 in order to enable a linear and an axial travel of the lead nut 8 along the lead screw 31.

In order to avoid an axial shift of the lead screw 31 in the distal direction, it may be equipped with a thrust bearing 35 that is mounted on the lead screw and abuts a step provided in the housing 32. An assembly of the motor reducer 30, the lead screw 31, and the thrust bearing 35 is mounted in the housing 32 until stop and fixed with a reducer portion of the motor reducer 30 by a gluing method.

The encoder 36 of the actuator 29 comprises a plate 37 that is fixed on the motor reducer and having a board 38 mounted thereon, the board is connected to the contacts of the motor reducer 30 and equipped with a means 39 for a releasable electrical connection in a form of saddles or flat platforms, and a magnet 40 is mounted on the rear shaft of the motor reducer 30. Therewith, at least two Hall sensors 41 are arranged on the board which are displaced radially from each other by 90 angle degrees. The magnet 40 is made in a form of a disc that rotates between the Hall sensors 41, and the plate 37 of the encoder 36 performs a centering function and comprises recesses for components of the releasable electrical connection 39. The actuator 29 is enabled to be electrically connected to a contact board 42 that is mounted in a frame 43 of the artificial wrist, then the motor reducer 30 is powered, the encoder 36 is powered, and pulses are transmitted from the Hall sensors 41 to the contact board 42. The encoder 36 reads a number of revolutions of the motor reducer 30 by means of the Hall sensors 41, the linear movement of the lead nut 8 on the lead screw 31 is calculated which is used to determine the bending angle of the mechanical finger 1, thereby providing the effective control of the latter.

The mechanical coupling between the mechanical finger 1 and the actuator 29, as well as between the actuator and the corresponding element of the artificial wrist 43, may be provided by means of releasable connection of their corresponding portions by any method and means known to a skilled person, e.g., by means of fixation screws 44.

Owing to the combination of the above-described elements of each of the technical solutions, it is enabled to create a structurally easy, reliable, and anatomical element of the artificial wrist that is characterized by a relatively easiness of manufacturing, provides the uniformity of transfer of forces from the actuator to the phalanxes of the mechanical finger, has the protection against unintentional damages due to the presence of the passing bending, and wherein it is enabled to easily replace the actuator and the finger as individual modules by performing easy operations.

Therefore, the group of inventions unites the claimed technical solutions by a single inventive concept, while providing their reliability, functionality, and modularity, and allows to create a final element for the artificial upper limb upon their interaction.

1. A mechanical finger for an artificial wrist, the finger comprising at least two movable phalanxes, a proximal one and a distal one, which are hingedly connected between each other and coupled by elements configured to modify a position of the distal phalanx relative to the proximal phalanx, which are driven by means of a rope, and the proximal phalanx is hingedly connected to an immovable base that is configured to be coupled to an electromechanical actuator and is equipped with an element that provides an interaction between the actuator and the rope, characterized in that a lead nut is used as the element that provides the interaction with the rope, the lead nut is arranged outside the hinge connection between the base and the proximal phalanx and is equipped with a fixation element to avoid its rotation, and the rope is secured on the lead nut and divided into two portions that are arranged symmetrically relative to a sagittal plane of the proximal phalanx and are laid on guides, wherein first guides are arranged on the base and represent bypass supports, while other guides are curved guides that are provided in the proximal phalanx around an axis of the hinge connection, and cavities are provided between the base and the proximal phalanx, the cavities being coupled to the curved guides so as they can receive loops of the rope, and a length of each loop equals a travel length of the lead nut, and the ends of the rope are fixed in the proximal phalanx.

2. The mechanical finger according to claim 1, characterized in that a rigid traction in a form of a lever having ends that are secured on rotation axes in the distal phalanx and in the base, and an elastic reverse force element in a form of a tension spring having ends that are secured in the distal phalanx and in the proximal phalanx, respectively, are used as the elements configured to modify the position of the distal phalanx relative to the proximal phalanx.

3. The mechanical finger according to claim 1, characterized in that a bearing that is mounted on an external surface of the nut is used as an element for fixation of the lead nut to avoid its rotation, wherein an axis of the bearing is perpendicular to an axis of the nut.

4. The mechanical finger according to claim 1, characterized in that the curved guides are made of metal.

5. The mechanical finger according to claim 1, characterized in that the bypass supports are made as roller supports.

6. The mechanical finger according to claim 1, characterized in that one or both of the distal or the proximal phalanx has an outer envelope.

7. A mechanical finger for an artificial wrist, the finger comprising at least two movable phalanxes, a proximal one and a distal one, which are hingedly connected between each other and coupled by elements configured to modify a position of the distal phalanx relative to the proximal phalanx, which are driven by means of a rope, and the proximal phalanx is hingedly connected to an immovable base that is configured to be coupled to an electromechanical actuator and is equipped with an element that provides an interaction between the actuator and the rope, characterized in that a lead nut is used as the element that provides the interaction with the rope, the lead nut is arranged outside the hinge connection between the base and the proximal phalanx and is equipped with a fixation element to avoid its rotation, and the rope is secured on the lead nut and divided into two portions that are arranged symmetrically relative to a sagittal plane of the proximal phalanx and are laid on guides, wherein first guides are arranged on the base and represent bypass supports, while second guides are arranged within the hinge connection that is formed by the base with a rotor mounted thereon, the rotor is immovably connected to the proximal phalanx, and the second guides represent bypass supports secured on the rotor, and a travel of the bypass supports of the rotor is limited by a chamber formed in the base, the chamber is configured to receive loops of the rope, and a length of each loop equals a travel length of the lead nut, and the ends of the rope are fixed in the base.

8. The mechanical finger according to claim 7, characterized in that a rigid traction in a form of a lever having ends that are secured on rotation axes in the distal phalanx and in the base, and an elastic reverse force element in a form of a tension spring having ends that are secured in the distal phalanx and in the proximal phalanx, respectively, are used as the elements configured to modify the position of the distal phalanx relative to the proximal phalanx.

9. The mechanical finger according to claim 7, characterized in that locations of the rope are closed by covers secured to the base.

10. The mechanical finger according to claim 7, characterized in that a bearing that is mounted on an external surface of the nut is used as an element for fixation of the lead nut to avoid its rotation, wherein an axis of the bearing is perpendicular to an axis of the nut.

11. The mechanical finger according to claim 7, characterized in that the bypass supports are made as roller supports.

12. The mechanical finger according to claim 7, characterized in that one or both of the distal or the proximal phalanx has an outer envelope.

13. An electromechanical actuator of the finger for an artificial wrist according to claim 1, the actuator comprising a motor reducer having a front shaft with a lead screw mounted thereon that is configured to be coupled to the lead nut of the mechanical finger, wherein a first portion of the motor reducer that is coupled to the lead screw is arranged in a housing, while an output portion of the motor reducer forms a chamber of the lead screw, the chamber is equipped with a guide for linear movement of the fixation element of the lead nut to avoid its rotation and is configured to be connected to the base of the finger, wherein an encoder is connected to a second portion of the motor reducer, the encoder comprising a plate that is mounted on the motor reducer, and a motor reducer board that is connected to contacts of the motor reducer is mounted on the plate, the board is equipped with at least two Hall sensors that are displaced radially from each other by a 90 degree angle, and a magnet that is mounted on a rear shaft, wherein the encoder plate is equipped with a means for a releasable electrical connection to corresponding contacts of the artificial wrist.

14. The electromechanical actuator according to claim 13, characterized in that the guide for the linear movement of the fixation element of the lead nut to avoid its rotation is a longitudinal groove.

15. The electromechanical actuator according to claim 13, characterized in that the means for the releasable electrical connection of the encoder board are saddles for needle contacts or flat platforms for spring-loaded contacts.
